(19) United States
(12) Patent Application Publication
(10) Pub. No.: US 2023/0203085 A1
(43) Pub. Date: Jun. 29, 2023

(54) MULTI-CONJUGATES COMPRISING MULTIPLE LIGANDS

(71) Applicants: MPEG LA, L.L.C., Chevy Chase, MD (US); City of Hope, Duarte, CA (US)

(72) Inventors: Marcin Tomasz Kortylewski, Monrovia, CA (US); Piotr Marek Swiderski, San Dimas, CA (US); Jonathan Miles Brown, Silver Spring, MD (US); Kristin K.H. Neuman, Larchmont, NY (US)

(21) Appl. No.: 17/901,674

(22) Filed: Sep. 1, 2022

Related U.S. Application Data
(60) Provisional application No. 63/240,310, filed on Sep. 2, 2021.

(51) Int. Cl. C07H 21/00; C12N 15/11

(52) U.S. Cl. CPC C07H 21/00; C12N 15/111; C12N 2310/11; C12N 2310/14; C12N 2310/3519; C12N 2330/30

(57) ABSTRACT

A multi-conjugate comprising two or more covalently linked biological subunits, wherein at least two of the subunits are terminally located targeting ligands. A pharmaceutical composition comprising a pharmaceutically acceptable excipient and a therapeutic agent that is the multi-conjugate is also disclosed. The multi-conjugate may be administered to a subject for providing treatment or prophylaxis against a disease or other medical condition.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD OF THE DISCLOSURE

The present disclosure relates to multi-conjugates that comprise a plurality of cell- or tissue-targeting ligands, methods of making such multi-conjugates, and methods of using them to improve uptake and delivery of multi-conjugates to target cells or tissues for the treatment or prevention of disease.

### BACKGROUND

Conjugates of multiple biological agents covalently linked together and further conjugated to a cell- or tissue-targeting ligand are of growing interest and importance in the field of biological pharmaceuticals. While certain conjugates have achieved clinical success when delivered to hepatocytes using the GalNAc ligand, cellular delivery and uptake remains a challenge for conjugates targeting cells or tissues other than hepatocytes and liver.

Therefore, there is a need for improved conjugates and delivery techniques in the field of biological therapeutics.

### SUMMARY

Various embodiments provide a multi-conjugate, a pharmaceutical composition comprising the multi-conjugates, methods of making them and methods of using them to provide treatment or prophylaxis against a disease or other medical condition as described below and summarized in the claims.

The disclosure provides a multi-conjugate comprising a plurality of covalently linked biological subunits (B), wherein at least two of the subunits are terminally located targeting ligands (L).

In some embodiments, the multi-conjugate comprises Structure 1: L-•-(B-•-)ₐ L, wherein each L is independently a targeting ligand, each B is independently a biological subunit, each-•-is independently a covalent linker; and a is an integer greater than or equal to 1.

In some embodiments, the multi-conjugate comprises Structure 2: L-•-O-•-(O-•-)ₐ O-•-L, wherein each L is independently a targeting ligand; each O is independently an oligonucleotide subunit; each-▪-is independently a covalent linker; and a is an integer greater than or equal to 0.

In some embodiments, the multi-conjugate comprises Structure 3: L-▪-O-□-(O-□-)ₐ O-▪-L, wherein each L is independently a targeting ligand; each O is independently an oligonucleotide subunit; each-□-is independently a cleavable covalent linker; each-▪-is independently a cleavable covalent linker that cleaves at a slower rate than-□-under human physiological conditions; a is an integer greater than or equal to 0.

In some embodiments, the multi-conjugate comprises Structure 4: L-•-(O-•-)ₐ (SSO-•-)_{b} (O-•-)_{c} L, wherein each L is independently a targeting ligand; each O is independently an oligonucleotide subunit; each SSO is independently a split-strand oligonucleotide subunit; each-•-is independently a covalent linker; a and c are each independently an integer greater than or equal to 0, and b is an integer greater than or equal to 1.

In some embodiments, the multi-conjugate comprises Structure 5: L-▪-O-□-(SSO-□-)_{b} O-▪-L, wherein each L is independently a targeting ligand; each O is independently an oligonucleotide subunit; each SSO is independently a split-strand oligonucleotide subunit; each-□-is independently a cleavable covalent linker; each-▪-is independently a cleavable covalent linker that cleaves at a slower rate than-□-under human physiological conditions; and b is an integer greater than or equal to 1.

In some embodiments, the multi-conjugate comprises Structure 6: L-•-EEM-•-(B-•-)ₐ EEM-•-L, wherein each L is independently a targeting ligand; each EEM is independently an endosomal escape moiety; each B is independently a biological subunit; each-•-is independently a covalent linker.

In some embodiments, the multi-conjugate comprises Structure 7: L-▪-EEM-▪-B-□-(B-□-)ₐ B-▪-EEM-▪-L; wherein each L is independently a targeting ligand; each EEM is independently an endosomal escape moiety; each-□-is independently a cleavable covalent linker; each-▪-is independently a cleavable covalent linker that cleaves at a slower rate.

The disclosure provides a pharmaceutical composition comprising a pharmaceutically acceptable excipient and a therapeutic agent that is a multi-conjugate described herein.

The disclosure provides a method of providing treatment or prophylaxis against a disease or other medical condition in a subject in need of medical treatment or prophylaxis, the method comprising administering to the subject an effective amount of the multi-conjugate described herein.

### BRIEF DESCRIPTION OF THE DRAWINGS

FIG. 1A presents a schematic representation of the synthesis of a dimer of the monomeric conjugate D19 ODN-miR146a.

FIG. 1B presents a schematic representation of the synthesis of a dimer of control compound D19 ODN-scrambled RNA.

FIG. 2 presents a schematic representation of the synthesis of a multi-conjugate comprising six subunits—two targeting ligands (D19 ODN), one positioned at each terminus of the multi-conjugate, two subunits of a miRNA mimic (miR-146a) and two subunits of a scrambled RNA (Scrambled). Sequences for the individual subunits and covalent linkers used in the synthesis of the multi-conjugate are presented on the right-hand side of the diagram.

FIG. 3A is a non-denaturing reverse phase Hplc chromatogram of Hetero-tetramer of active C-miR146a.

FIG. 3B is an ESI-MS Spectrum of Hetero-tetramer of active C-miR146a.

FIG. 4A presents the results of Time-dependent internalization of Cy3-labeled oligonucleotide monomers or tetramers by mouse macrophages.

FIG. 4B presents the intracellular uptake of Cy3-labeled C-miR146 monomeric or tetrameric oligonucleotides by mouse macrophages.

FIG. 5 presents organ biodistribution of monomeric and tetrameric C-miR146 oligonucleotides in mice.

FIG. 6 presents cellular biodistribution of monomeric and tetrameric C-mir146 oligonucleotides in mice.

### DETAILED DESCRIPTION

The term “multi-conjugate” as used herein has its ordinary meaning as understood by those skilled in the art. It refers to compounds that comprise two or more subunits joined to one another by a covalent linker, wherein each of the subunits is, independently, a biological agent.

The terms “biological agent” or “biological subunit” as used herein have their ordinary meaning as understood by those skilled in the art. They refer to chemical entities that are biologically active or inert when delivered into a cell or organism. In many instances, a biological agent will produce a biological effect or activity within the cell or organism to which it is delivered; and oftentimes the biological effect or activity is detectable or measurable. In other instances, a biological agent may be selected to augment or enhance the biological effect or activity of another biological agent with which it is delivered. Examples of biological agents include a nucleic acid, oligonucleotide, amino acid, peptide, protein, lipid, carbohydrate, carboxylic acid, vitamin, steroid, lignin, small molecule, organometallic compound, or a derivative of any of the foregoing.

The term “targeting ligand” as used herein has its ordinary meaning as understood by those skilled in the art. It refers to any of a variety of atoms, molecules or compounds that bind with specificity or selectivity to a cell surface receptor or other feature of a cell or tissue and thereby enable “targeted” delivery of any biological agents that may be conjugated to the targeting ligand.

The term “human physiological conditions” as used herein has its ordinary meaning as understood by those skilled in the art. It refers to the conditions that cells in the human body experience or function under. For example, human physiological conditions include an aqueous solution at about 37° C. and at a pH of about 7.4.

### Multi-Conjugates

The disclosure provides various multi-conjugates comprising a plurality of covalently linked biological subunits for improved pharmacokinetic and pharmacodynamic effects.

In some embodiments the molecular weight and/or size of the multi-conjugate is configured to produce a multi-conjugate having increased serum half-life when administered in vivo.

The disclosure provides for multi-conjugates comprising a variety of biological subunits. The multi-conjugates may be “homo” conjugates, wherein each biological subunit is the same; or the multi-conjugates may be “hetero” conjugates, wherein one or more biological subunits are different.

In the context of a hetero multi-conjugate, the multi-conjugate may be configured to deliver—in a single compound —the various biological subunits in precise stoichiometric and/or positional control.

The disclosure provides for multi-conjugates comprising cleavable covalent linkages such that the individual subunits may be liberated upon or after delivery to the target cell or tissue, or to a particular intracellular compartment.

In some embodiments, the multi-conjugate is configured to enable liberation of its various subunits at different times and/or under different conditions through selection of the properties of the covalent linkers employed in the multi-conjugate. For example, in an embodiment a combination of —[(CH₂)₃PO₂]₅— linkers and disulfide-containing linkers are employed in a multi-conjugate. The disulfide containing linkers will cleave relatively rapidly in the reductive conditions of the cytosol. In contrast, the dialkyl phosphodiester linkages will be cleaved more slowly over time by nucleases.

The disclosure provides a multi-conjugate comprising a plurality of covalently linked biological subunits (B), wherein at least two of the subunits are terminally located targeting ligands (L).

In some embodiments, the multi-conjugate comprises the following Structure 1:

L-•-(B-•-)ₐL (Structure 1);
wherein each L is independently a targeting ligand, each B is independently a biological subunit, each-•-is independently a covalent linker; and a is an integer greater than or equal to 1, or a is an integer from 1 to 25; or a is 1, 2, 3, 4, 5, 6, 7, 8, 9, 10, 11 or 12. In some embodiments, B is an oligonucleotide subunit. In some embodiments, a is 1. In some embodiments, the covalent linker may be a cleavable covalent linker.

In some embodiments, the multi-conjugate comprises the following Structure 2:

L-•-O-•-(O-•-)ₐO-•-L (Structure 2);

wherein each L is independently a targeting ligand; each O is independently an oligonucleotide subunit; each-•-is independently a covalent linker; and a is an integer greater than or equal to 0; or a is an integer from 0 to 20; or a is 0, 1, 2, 3, 4, 5, 6, 7, 8, 9 or 10. In some embodiments, the covalent linker may be a cleavable covalent linker.

In some embodiments, the multi-conjugate comprises the following Structure 3:

L-▪-O-□-(O-□-)ₐO-▪-L (Structure 3);
wherein each L is independently a targeting ligand; each O is independently an oligonucleotide subunit; each-□-is independently a cleavable covalent linker; each-▪-is independently a cleavable covalent linker that cleaves at a slower rate than-□-under human physiological conditions; a is an integer greater than or equal to 0, or a is an integer from 0 to 20, or a is 0, 1 2, 3, 4 5, 6, 7, 8, 9 or 10. In some embodiments, each-▪-is —[(CH₂)₃PO₂]₅, and each-□-is a cleavable covalent linker derived from dithiobismaleimidoethane (DTME).

In some embodiments, at least one of the biological subunits B is a double-stranded oligonucleotide subunit comprised of two complementary strands, each comprising a chain of nucleic acids, and wherein one of the strands contains a break in its chain (i.e., a split-strand oligonucleotide subunit).

In some embodiments, the multi-conjugate comprises the following Structure 4:

L-•-(O-•-)ₐ(SSO-•-)_{b}(O-•-)_{c}L (Structure 4);
wherein each L is independently a targeting ligand; each O is independently an oligonucleotide subunit; each SSO is independently a split-strand oligonucleotide subunit; each-•-is independently a covalent linker; a and c are each independently an integer greater than or equal to 0; or a and c are each independently an integer from 0 to 20; or a and c are each independently 0, 1, 2, 3, 4, 5, 6, 7, 8, 9, 10, 11 or 12; and b is an integer greater than or equal to 1, or b is an integer from 1 to 20; or b is 1, 2, 3, 4, 5, 6, 7, 8, 9, 10, 11 or 12. In some embodiments, the covalent linker may be a cleavable covalent linker.

In some embodiments, the multi-conjugate comprises the following Structure 5:

L-▪-O-□-(SSO-□-)_{b}O-▪-L (Structure 5);

wherein each L is independently a targeting ligand; each O is independently an oligonucleotide subunit; each SSO is independently a split-strand oligonucleotide subunit; each-□-is independently a cleavable covalent linker; each-▪-is independently a cleavable covalent linker that cleaves at a slower rate than-□-under human physiological conditions; and b is an integer greater than or equal to 1; or b is an integer from 1 to 12; or b is 1, 2, 3, 4, 5, 6, 7, 8, 9 or 10.

In some embodiments, the multi-conjugate comprises the following Structure 6:

L-•-EEM-•-(B-•-)ₐEEM-•-L (Structure 6);

wherein each L is independently a targeting ligand; each EEM is independently an endosomal escape moiety; each B is independently a biological subunit; each-•-is independently a covalent linker; a is an integer greater than or equal to 1; or a is an integer from 1 to 25; or a is 1, 2, 3, 4, 5, 6, 7, 8, 9, 10, 11 or 12. In some embodiments, each-•-is independently a cleavable covalent linker. In some embodiments, each B independently comprises an oligonucleotide, peptide, protein, lipid, carbohydrate, carboxylic acid, steroid, vitamin, small molecule organic compound, organometallic compound, or inorganic compound.

In some embodiments, the multi-conjugate comprises the following Structure 7:

L-▪-EEM-▪-B-□-(B-□-)ₐB-▪-EEM-▪-L (Structure 7);

wherein each L is independently a targeting ligand; each EEM is independently an endosomal escape moiety; each B is independently a biological subunit; each-□-is independently a cleavable covalent linker; each-▪-is independently a cleavable covalent linker that cleaves at a slower rate than-□-under human physiological conditions; and a is an integer greater than or equal to 0; or a is an integer from 0 to 25; or a is 0, 1, 2, 3, 4, 5, 6, 7, 8, 9, 10, 11 or 12. In some embodiments, each B independently comprises an oligonucleotide, peptide, protein, lipid, carbohydrate, carboxylic acid, steroid, vitamin, small molecule organic compound, organometallic compound, or inorganic compound. In some embodiments, each B in the multi-conjugate is independently an oligonucleotide subunit.

### Targeting Ligands

In some embodiments, the multi-conjugate comprises at least two terminally located targeting ligands. In some embodiments, at least one targeting ligand L in the multi-conjugate is a CpG-containing deoxy-oligonucleotide (ODN). In some embodiments, all of the targeting ligands L in the multi-conjugate are a CpG-containing ODN.

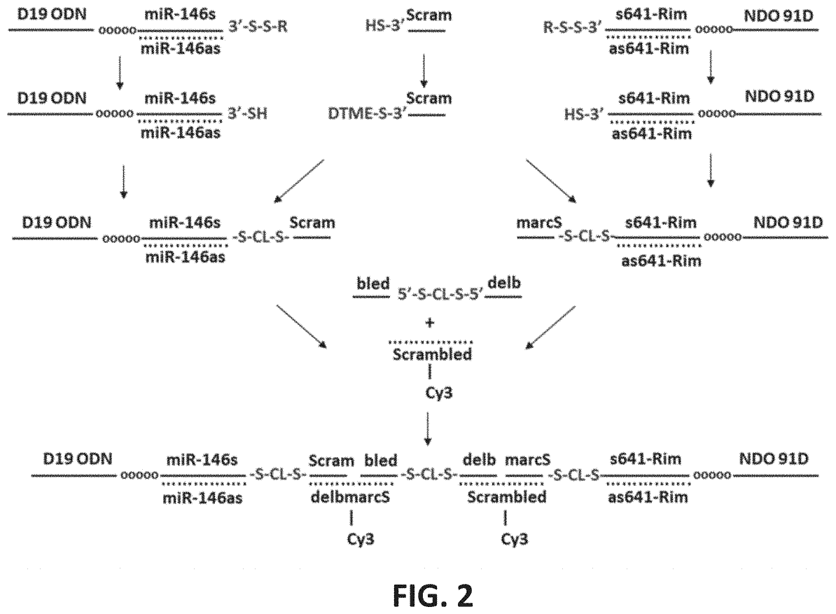

In some embodiments, the targeting ligands are single-stranded deoxy-oligonucleotides containing unmethylated CpG motifs, which are internalized through a scavenger receptor-dependent mechanism and activate intracellular Toll-like Receptor 9 (TLR 9) in the target cell (which, e.g., may be myeloid immune cells or B cells). In some such embodiments, the ligand comprises deoxy-oligonucleotide D19 (D19 ODN), having the sequence 5′-G*G*TGCATCGATGCAGG*G*G*G*G-3′, where * is a phosphorothioate internucleotide linkage (see sequence provided in FIG. 2). See also Su Y-L et al., BLOOD, 135 (3) 2020, which is incorporated herein by reference in its entirety.

### Biological Subunits

In some embodiments, the biological subunit (B) may independently comprise an oligonucleotide, peptide, protein, lipid, carbohydrate, carboxylic acid, steroid, vitamin, small molecule organic compound, organometallic compound, or inorganic compound. In some embodiments, the biological subunit may be an oligonucleotide. In some embodiments, at least one oligonucleotide subunit O in the multi-conjugate is siRNA, saRNA, miRNA, or an antisense oligonucleotide. In some embodiments, all of the oligonucleotide subunits O in the multi-conjugate are miRNA mimic.

In some embodiments, the multi-conjugate comprises one or more oligonucleotide subunits of a microRNA (miRNA) mimic. In some embodiments, the miRNA mimic is miR-146a, which comprises sequence 5′-CCCAUGGAAUUCAGUUCUCAaA-3′ as a passenger strand, wherein “a” signifies 2′-MeO modified Adenine, and 5′-UGAGAACUGAAUUCCAUGGGUU-3′ as guide strand.

miR-146a is a negative feedback inhibitor of NF-κB with tumor suppressor activity. NF-κB is a key regulator of inflammation and cancer progression, with an important role in leukemogenesis. See Mehta & Baltimore, NAT. REV. IMMUNOL. 2016; Starczynowski, NAT. MED. 2010.

In some embodiments, at least one split-strand oligonucleotide subunit SSO in the multi-conjugate is siRNA, saRNA, or miRNA.

In some embodiments, the multi-conjugate comprises Structure 3, wherein each L is D19 ODN; each O is miR-146a; each-▪-is —[(CH₂)₃PO₂]₅—;-□-is a cleavable covalent linker derived from dithiobismaleimidoethane (DTME); and a is 0.

In some embodiments, the multi-conjugate comprises Structure 5, wherein each L is D19 ODN; each O is miR-146a; each-▪-is —[(CH₂)₃PO₂]₅—; each-□-is a covalent linker derived from dithiobismaleimidoethane (DTME); and b is 2.

A monomeric conjugate of D19 ODN and miR-146a is disclosed in Su Y-L et al., BLOOD, 135 (3) 2020, which is incorporated herein by reference in its entirety.

### Pharmaceutical Compositions and Medicaments

The disclosure provides for a pharmaceutical composition comprising a pharmaceutically acceptable excipient and a therapeutic agent that is a multi-conjugate as described herein, including but not limited to any of Structures 1 to 7, or as recited in any of claims 1 to 31, which follow. In some embodiments, the pharmaceutical composition may further comprise a second therapeutic agent. In some embodiments, the second therapeutic agent is an anti-tumor or anti-cancer agent, cytotoxic agent, cytostatic agent, anti-inflammatory agent, analgesic, anti-infective agent, growth inhibitory agent, immunogenic agent, immunomodulatory agent, or chemokine.

The disclosure further provides a multi-conjugate for use in the manufacture of a medicament, wherein the multi-conjugate comprises a plurality of covalently linked biological subunits and at least two of the subunits are terminally located targeting ligands, including but not limited to of any of Structures 1 to 7, or as recited in any of claims 1 to 31, which follow.

As used herein, pharmaceutical compositions include compositions of matter, other than foods, that contain one or more active pharmaceutical ingredients that can be used to prevent, diagnose, alleviate, treat, or cure a disease. Similarly, the various compounds or compositions according to the disclosure should be understood as including embodiments for use as a medicament and/or for use in the manufacture of a medicament.

As used herein, an excipient can be a natural or synthetic substance formulated alongside the active ingredient. Excipients can be included for the purpose of long-term stabilization, increasing volume (e.g., bulking agents, fillers, or diluents), or to confer a therapeutic enhancement on the active ingredient in the final dosage form, such as facilitating drug absorption, reducing viscosity, or enhancing solubility. Excipients can also be useful in manufacturing and distribution, for example, to aid in the handling of the active ingredient and/or to aid in vitro stability (e.g., by preventing denaturation or aggregation). As will be understood by those skilled in the art, appropriate excipient selection can depend upon various factors, including the route of administration, dosage form, and active ingredient(s).

The pharmaceutical composition can be delivered locally or systemically, and the administrative route for pharmaceutical compositions of the disclosure can vary according to application. Administration is not necessarily limited to any particular delivery system and may include, without limitation, parenteral (including subcutaneous, intravenous, intramedullary, intraarticular, intramuscular, intraperitoneal, intraparenchymal, intracerebroventricular, and intrathecal, cisternal and lumbar), rectal, topical, transdermal, or oral. In some embodiments, the multi-conjugate or pharmaceutical composition is administered to the subject intravenously. Administration to an individual may occur in a single dose or in repeat administrations, and in any of a variety of physiologically acceptable salt forms, and/or with an acceptable pharmaceutical carrier and/or additive or adjuvant as part of a pharmaceutical composition. Physiologically acceptable formulations and standard pharmaceutical formulation techniques, dosages, and excipients are well known to persons skilled in the art (see, e.g., Physicians' Desk Reference (PDR®) 2005, 59th ed., Medical Economics Company, 2004; and Remington: The Science and Practice of Pharmacy, eds. Gennado et al. 21st ed., Lippincott, Williams & Wilkins, 2005).

Pharmaceutical compositions can include an effective amount of the multi-conjugate compound or composition according to the disclosure. As used herein, effective amount can be a concentration or amount that results in achieving a particular purpose, or an amount adequate to cause a change, for example in comparison to a placebo. Where the effective amount is a therapeutically effective amount, it can be an amount adequate for therapeutic use, for example an amount sufficient to prevent, diagnose, alleviate, treat, or cure a disease or condition. An effective amount can be determined by methods known in the art. An effective amount can be determined empirically, for example by human clinical trials. Effective amounts can also be extrapolated from one animal (e.g., mouse, rat, monkey, pig, dog) for use in another animal (e.g., human), using conversion factors known in the art. See, e.g., Freireich et al., Cancer Chemother Reports 50(4):219-244 (1966).

### Methods of Using Products Comprising the Multi-Conjugate

The present disclosure also relates to methods of using compounds containing the multi-conjugate as described herein in various applications, including but not limited to delivery to cells in vitro or in vivo for the purpose of modulating gene expression, biological research, treating or preventing medical conditions, and/or to produce new or altered phenotypes.

In an embodiment, the disclosure provides a method of treating or prophylaxis against a disease or other medical condition in a subject in need of medical treatment or prophylaxis by administering to the subject an effective amount of multi-conjugate as disclosed herein, including but not limited to of any of Structures 1 to 7, or as recited in any of claims 1 to 31 or a pharmaceutical composition comprising an effective amount of multi-conjugate.

In some embodiments, the disease is cancer, infectious disease, or inflammatory disorder. In some embodiments, the disease is hematopoietic cancer, a myeloproliferative disorder, myeloma, or myeloid leukemia. In some embodiments, the medical condition is sepsis or cytokine release syndrome.

As used herein, a “subject” includes, but is not limited to, mammals, such as primates, rodents, and agricultural animals. Examples of a primate subject includes, but is not limited to, a human, a chimpanzee, and a rhesus monkey. Examples of a rodent subject includes, but is not limited to, a mouse and a rat. Examples of an agricultural animal subject includes, but is not limited to, a cow, a sheep, a lamb, a chicken, and a pig

### Methods for Treating Subjects

The disclosure provides a method for treating a subject in need of treatment to ameliorate, cure, or prevent the onset of a disease or disorder, the method comprising administering to the subject an effective amount of the multi-conjugate as described herein, including but not limited to any of Structures 1 to 7, or as recited in any of claims 1 to 31, which follow.

The disclosure provides a method of treating a disease or condition in a subject comprising the step of administering to the subject an effective amount of a pharmaceutical composition comprising a multi-conjugate as described herein, including but not limited to any of Structures 1 to 7, or as recited in any of claims 1 to 31, which follow.

The following Examples are illustrative and not restrictive. Many variations of the technology will become apparent to those of skill in the art upon review of this disclosure. The scope of the technology should, therefore, be determined not with reference to the Examples, but instead should be determined with reference to the appended claims along with their full scope of equivalents.

### Example 1

### Synthesis of Dimer of Active C-miR146a

C-miR146a active (C-miR146a is a D19 ODN conjugated to miR0146a) was prepared as described previously (Su Y-L et al; Blood, 135 (3), 2020) to yield 1.2 μmol of title compound in 99.0% purity. C-miR146a control was prepared as described previously (Su Y-L et al; Blood, 135 (3), 2020) to yield 1.2 μmol of title compound in 98.7% purity.

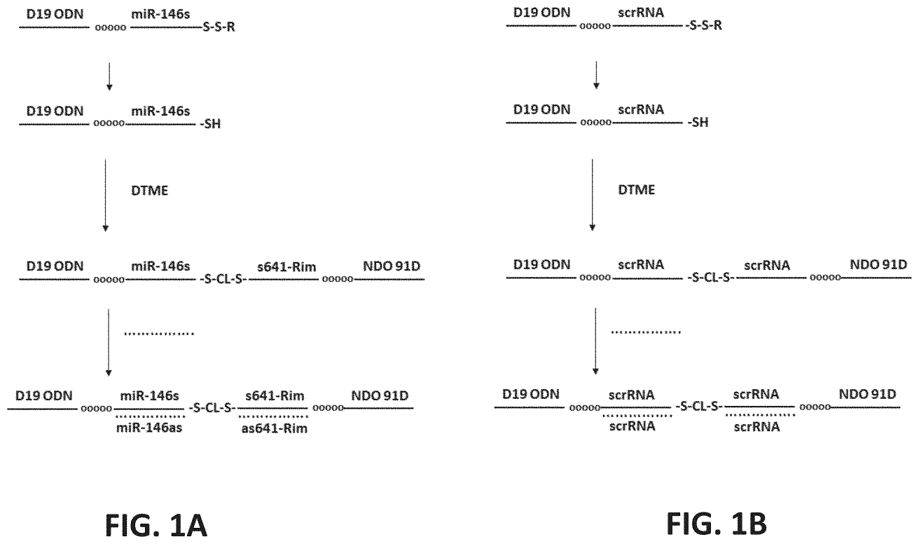

Dimer of active C-miR146a was prepared according to the first reaction scheme in FIG. 1A. Intact C-miR146a passenger strand was prepared on the synthesizer with the addition of a 3′-terminal disulfide. After unblocking and purification the disulfide was cleaved by treatment with dithiothreitol. The resulting 3′-thiolated material was added to 0.5 equivalents of dithioethylmaleimide (DTME) to dimerize the passenger strand which was then annealed with 2 equivalents of miR146a guide strand to yield 490 nmol of the title compound in 85.2% purity.

### Synthesis of Dimer of C-scrRNA control

Dimer of C-scrRNA control was prepared according to the second reaction scheme shown in FIG. 1B. Intact C-scrRNA control strand was prepared on the synthesizer with the addition of a 3′-terminal disulfide. After unblocking and purification the disulfide was cleaved by treatment with dithiothreitol. The resulting 3′-thiolated material was added to 0.5 equivalents of dithioethylmaleimide (DTME) to dimerize the passenger strand which was then annealed with 2 equivalents of scrambled guide strand to yield: 995 nmol of the title compound in 90.6% purity.

### Example 2

### Synthesis of Hetero-Tetramer of Active C-miR146a

Hetero-tetramer of active C-miR146a was prepared according to the reaction scheme shown in FIG. 2. Intact C-miR146a passenger strand was prepared on the synthesizer with the addition of a 3′-terminal disulfide as for the dimer of C-miR146a (which is described above in Example 1). After unblocking and purification the disulfide was cleaved by treatment with dithiothreitol. In this case the liberated thiol was treated with the 3′-monoDMTE derivative of the 3′-half of a fully protected scrambled passenger strand (“Scram”) to yield an asymmetric single stranded dimer. This material was then treated firstly with 1 equivalent of full length fully protected scrambled guide strand (labeled with Cy3) and then secondly with 0.5 equivalent of the symmetrical 5′-DTME linked dimer of the 5′-half of a fully protected scrambled passenger strand (“bled”). The resulting partially double stranded hetero-tetramer was then annealed to yield 1.2 umol of the title compound in 78.4% purity.

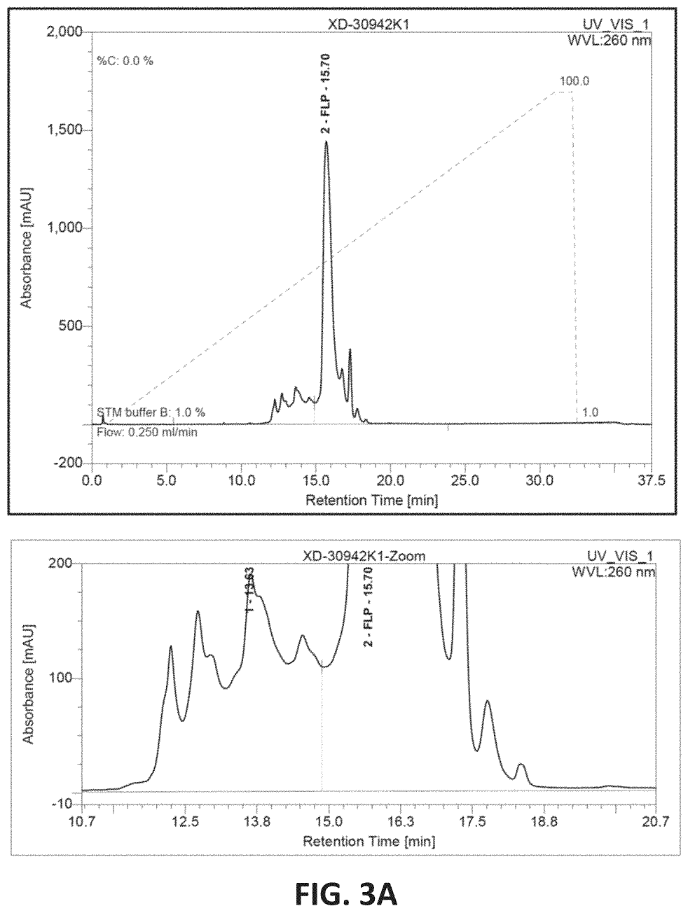

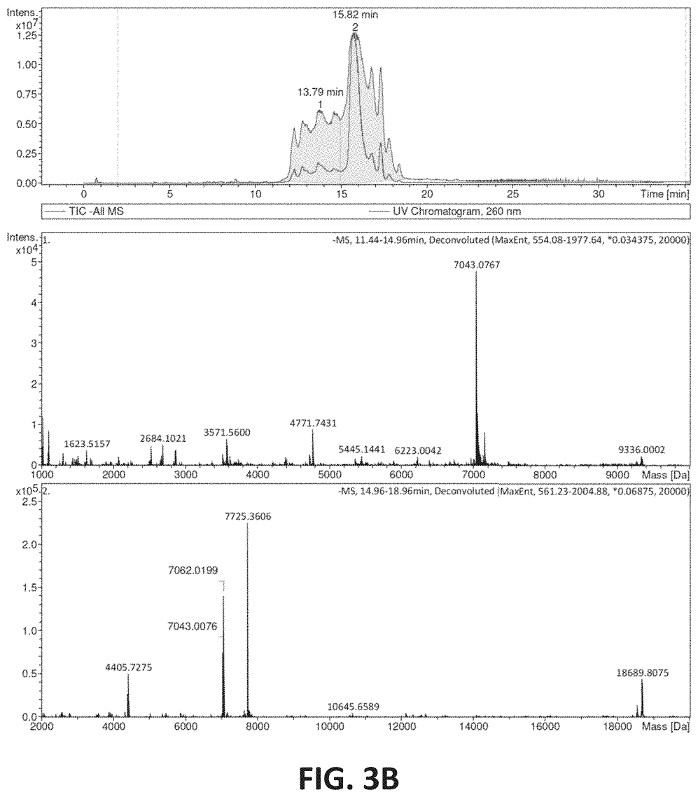

Non-denaturing Reverse Phase Hplc chromatogram of Hetero-tetramer of active C-miR146a is shown in FIG. 3A. ESI-MS Spectrum of Hetero-tetramer of active C-miR146a is shown in FIG. 3B.

### Synthesis of Hetero-Tetramer of C-scrRNA Control

Hetero-tetramer of C-scrRNA control was also prepared by the procedure in the reaction scheme shown in FIG. 2, with intact C-scrRNA control being substituted for C-miR146a. This yielded 1.2 μmol of the control compound in 82.3% purity.

Sequences

| | |
|---|---|
| D19 ODN: | 5′-G*G*TGCATCGATGCAGG*G*G*G* |
| | G-3′ |
| miR146a passenger: | CCCAUGGAAUUCAGUUCUCAaA-3′ |
| miR146a guide: | 5′-UGAGAACUGAAUUCCAUGGGUU-3′ |
| Scram passenger: | 5′-AfaUfaCfaCfgCfcAf*a-3′ |
| bled passenger: | 5′-Af*uUfuAfgCfcUfu-3′ |
| Scrambled guide: | 5′-Gf*gCfgUfgUfaUfuAfaGfgCfu |
| | AfaAfuCf*u-3′ |
| ooooo = —[(CH₂)₃—PO₂]₅— | |
| DTME = dithioethyl- | |
| maleimide | |
| -S-CL-S- = alkylthio- | |
| DTME-thioalkyl | |
| X = 2′-deoxy X | |
| x = 2′-MeO-X | |
| Xf = 2′deoxy-2′- | |
| fluoro-X | |
| * = phosphorothioate | |

### Example 3

### Internalization of Monomeric and Tetrameric C-miR146a Oligonucleotides by Target Mouse Macrophages

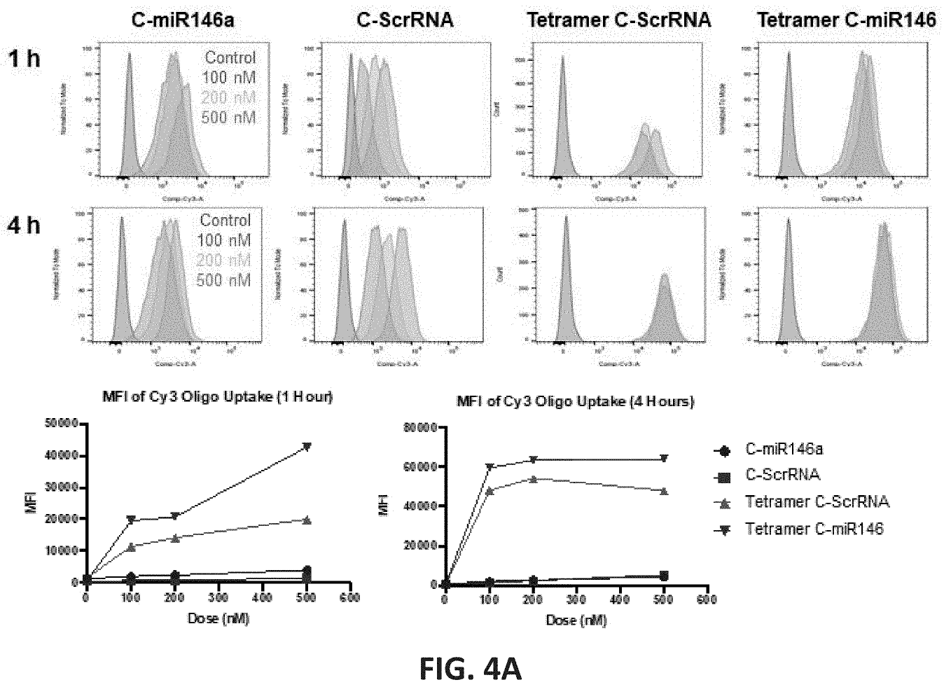

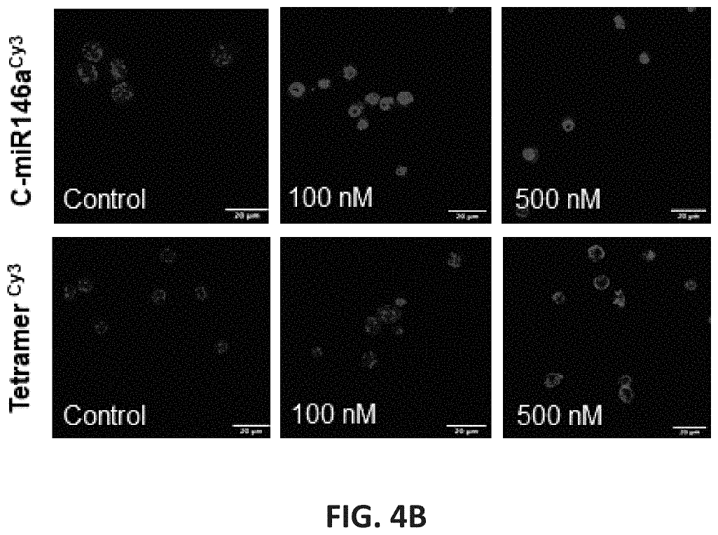

FIG. 4A shows the results of Time-dependent internalization of Cy3-labeled oligonucleotide monomers or tetramers by mouse RAW 264.7 macrophages after 1- or 4-hours incubation as measured using flow cytometry. Shown are the histogram overlays (left four panels) and the graphs summarizing difference in the mean fluorescent intensity (MFI) for various treatments. FIG. 4B demonstrates Intracellular uptake of Cy3-labeled C-miR146 monomeric or tetrameric oligonucleotides by mouse macrophages. Cells were incubated with 100 or 500 nM of fluorescently labeled oligonucleotides for 4 hours. The intracellular localization of oligonucleotides (red) and nuclei (blue, stained using DAPI) was detected using phase contrast and confocal microscopy; scale bar=20 μm; DAPI, 4, 6 diamidino-2-phenylindole.

### Example 4

### Monomer and Tetramer Biodistribution in Mice

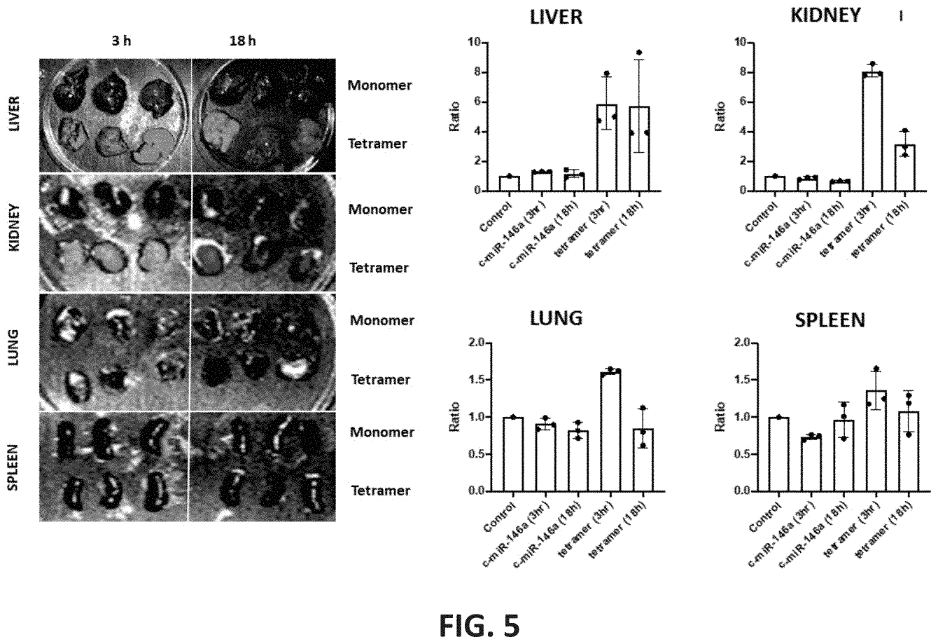

C57/BL6 mice were injected intravenously (IV) using fluorescently labeled monomeric (5 mg/kg) or tetrameric (17 mg/kg)C-miR146a oligonucleotides representing the equivalent molar amount of 230 nmoles. After 3 or 18 hours, mice were euthanized to harvest organs such as spleen, lung, liver and kidney. The fluorescent signal accumulated in various organs was compared using Lagos equipment. Shown in FIG. 5 are images collected from organs collected from 3 individual mice (A) and the signal quantification (B).

### Example 5

### Cellular Biodistribution of Monomeric and Tetrameric C-miR146a Oligonucleotides in Mice

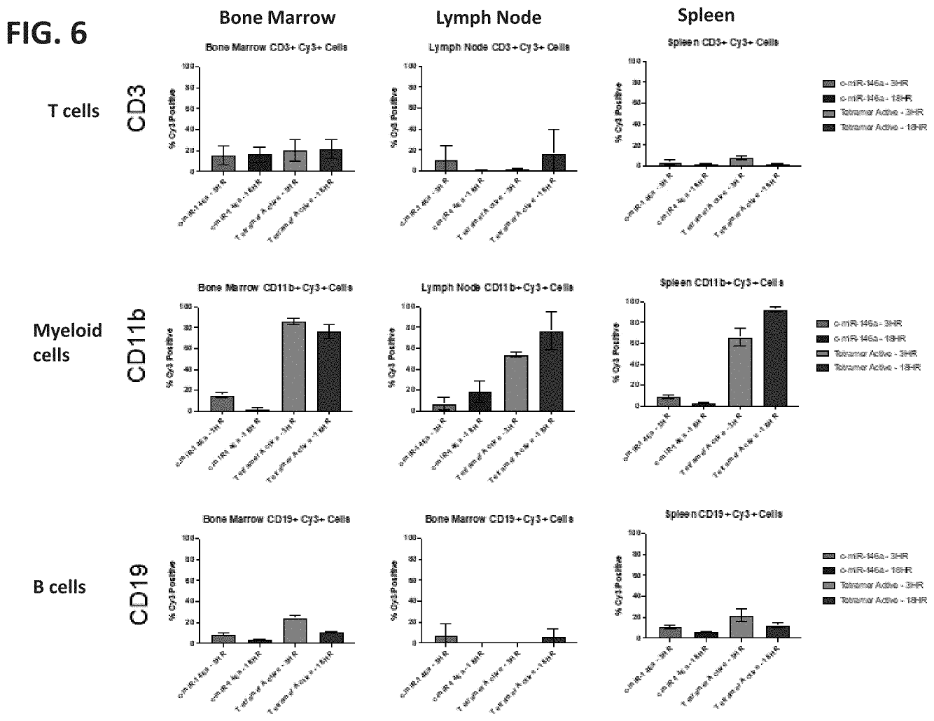

C57/BL6 mice were injected intravenously (IV) using fluorescently labeled monomeric (5 mg/kg) or tetrameric (17 mg/kg)C-miR146a oligonucleotides representing the equivalent molar amount of 230 nmoles. After 3 or 18 hours, mice were euthanized to harvest organs such as bone marrow, lymph nodes and spleen and prepare single cell suspensions. The uptake of tested oligonucleotides was assessed in myeloid cells (CD11b+), B cells (CD19+) and T cells (CD3+) using flow cytometry after staining with specific antibodies. Shown in FIG. 6 are bar graphs summarizing data from 3 individual mice.

1. A multi-conjugate comprising a plurality of covalently linked biological subunits (B), wherein at least two of the subunits are terminally located targeting ligands (L).

2. The multi-conjugate of claim 1, wherein the multi-conjugate comprises Structure 1:
L-•-(B-•-)ₐL (Structure 1)
wherein:
each L is independently a targeting ligand;
each B is independently a biological subunit, which independently comprises an oligonucleotide, peptide, protein, lipid, carbohydrate, carboxylic acid, steroid, vitamin, small molecule organic compound, organometallic compound, or inorganic compound;
each-•-is independently a covalent linker; and
a is an integer greater than or equal to 1.

3. The multi-conjugate of claim 2, wherein B is an oligonucleotide subunit and a is 1.

4. The multi-conjugate of claim 1, wherein the multi-conjugate comprises Structure 2:
L-•-O-•-(O-•-)ₐO-•-L (Structure 2)
wherein:
each L is independently a targeting ligand;
each O is independently an oligonucleotide subunit;
each-•-is independently a covalent linker; and
a is an integer greater than or equal to 0.

5. (canceled)

6. The multi-conjugate of claim 1, wherein the multi-conjugate comprises Structure 3:
L-▪-O-□-(O-D□-)ₐO-▪-L (Structure 3)
wherein:
each L is independently a targeting ligand;
each O is independently an oligonucleotide subunit;
each-□-is independently a cleavable covalent linker each-□-is independently a cleavable covalent linker that cleaves at a slower rate than-□-under human physiological conditions;
a is an integer greater than or equal to 0.

7. The multi-conjugate of claim 1, wherein at least one targeting ligand L in the multi-conjugate is a CpG-containing deoxy-oligonucleotide (ODN).

8. (canceled)

9. The multi-conjugate of claim 7, wherein the CpG-containing ODN comprises the sequence 5′-G*G*TGCATCGATGCAGG*G*G*G*G-3′ (D19 ODN), wherein * is a phosphorothioate internucleotide linkage.

10. The multi-conjugate of claim 3, wherein at least one oligonucleotide subunit O in the multi-conjugate is siRNA, saRNA, miRNA, or an antisense oligonucleotide.

11. The multi-conjugate of claim 3, wherein at least one oligonucleotide subunit O in the multi-conjugate is miRNA mimic.

12. (canceled)

13. (canceled)

14. The multi-conjugate of claim 6, wherein:
each L is D19 ODN;
each O is miR-146a;
each-▪-is —[(CH₂)₃PO₂]₅—;
-□-is a cleavable covalent linker derived from dithiobismaleimidoethane (DTME); and
a is 0.

15. The multi-conjugate of claim 1, wherein at least one of the biological subunits B is a double-stranded oligonucleotide subunit comprised of two complementary strands each comprising a chain of nucleic acids, and wherein one of the strands contains a break in its chain (a split-strand oligonucleotide subunit).

16. The multi-conjugate of claim 15, wherein the multi-conjugate comprises Structure 4:
L-•-(O-•-)ₐ(SSO-•-)_{b}(O-•-)_{c}L (Structure 4)
wherein:
each L is independently a targeting ligand;
each O is independently an oligonucleotide subunit;
each SSO is independently a split-strand oligonucleotide subunit;
each-•-is independently a covalent linker;
a and c are each independently an integer greater than or equal to 0; and
b is an integer greater than or equal to 1.

17. (canceled)

18. The multi-conjugate of claim 16, wherein the multi-conjugate comprises Structure 5:
L-▪-O-□-(SSO-□-)_{b}O-▪-L (Structure 5)
wherein:
each L is independently a targeting ligand;
each O is independently an oligonucleotide subunit;
each SSO is independently a split-strand oligonucleotide subunit;
each-□-is independently a cleavable covalent linker;
each-▪-is independently a cleavable covalent linker that cleaves at a slower rate than-□-under human physiological conditions; and
b is an integer greater than or equal to 1.

19. The multi-conjugate of claim 15, wherein at least one targeting ligand L in the multi-conjugate is a CpG-containing deoxy-oligonucleotide (ODN).

20. (canceled)

21. The multi-conjugate of claim 19, wherein the CpG-containing ODN comprises the sequence 5′-G*G*TGCATCGATGCAGG*G*G*G*G-3′ (D19 ODN), wherein * is a phosphorothioate internucleotide linkage.

22. The multi-conjugate of claim 16, wherein the multi-conjugate comprises at least one oligonucleotide subunit O which is siRNA, saRNA, miRNA, or an antisense oligonucleotide.

23. The multi-conjugate of claim 16, wherein at least one split-strand oligonucleotide subunit SSO in the multi-conjugate is siRNA, saRNA, or miRNA.

24. The multi-conjugate of claim 16, wherein the multi-conjugate comprises at least one oligonucleotide subunit O which is miRNA mimic.

25. (canceled)

26. (canceled)

27. The multi-conjugate of claim 18, wherein:
each L is D19 ODN;
each O is miR-146a;
each-▪-is —[(CH₂)₃PO₂]₅—;
each-□-is a covalent linker derived from dithiobismaleimidoethane (DTME); and
b is 2.

28. The multi-conjugate of claim 1, wherein the multi-conjugate comprises Structure 6:
L-•-EEM-•-(B-•-)ₐEEM-•-L (Structure 6)
wherein:
each L is independently a targeting ligand;
each EEM is independently an endosomal escape moiety;
each B is independently a biological subunit, which independently comprises an oligonucleotide, peptide, protein, lipid, carbohydrate, carboxylic acid, steroid, vitamin, small molecule organic compound, organometallic compound, or inorganic compound;
each-•-is independently a covalent linker;
a is an integer greater than or equal to 1.

29. (canceled)

30. The multi-conjugate of claim 1, wherein the multi-conjugate comprises Structure 7:
L-▪-EEM-▪-B-□-(B-□-)ₐB-▪-EEM-▪-L (Structure 7)
wherein:
each L is independently a targeting ligand;
each EEM is independently an endosomal escape moiety;
each B is independently a biological subunit, which independently comprises an oligonucleotide, peptide, protein, lipid, carbohydrate, carboxylic acid, steroid, vitamin, small molecule organic compound, organometallic compound, or inorganic compound;
each-□-is independently a cleavable covalent linker;
each-▪-is independently a cleavable covalent linker that cleaves at a slower rate than-□-under human physiological conditions; and
a is an integer greater than or equal to 0.

31. The multi-conjugate of claim 28, wherein each B in the multi-conjugate is independently an oligonucleotide subunit.

32. A pharmaceutical composition comprising a pharmaceutically acceptable excipient and a therapeutic agent that is a multi-conjugate according to claim 1.

33. The pharmaceutical composition of claim 32, further comprising a second therapeutic agent.

34. The pharmaceutical composition of claim 33, wherein the second therapeutic agent is an anti-tumor or anti-cancer agent, cytotoxic agent, cytostatic agent, anti-inflammatory agent, analgesic, anti-infective agent, growth inhibitory agent, immunogenic agent, immunomodulatory agent, or chemokine.

35. A method of providing treatment or prophylaxis against a disease or other medical condition in a subject in need of medical treatment or prophylaxis, the method comprising administering to the subject an effective amount of the multi-conjugate according to claim 1.

36-41. (canceled)

42. The method of claim 35, wherein the multi-conjugate or pharmaceutical composition is administered to the subject intravenously.
